(19) United States
(12) Patent Application Publication
(10) Pub. No.: US 2011/0105919 A1
(43) Pub. Date: May 5, 2011

(54) MEDICAL DEVICE

(76) Inventor: MOHAMMED NAJI, Riverside, CA (US)

(21) Appl. No.: 12/610,218

(22) Filed: Oct. 30, 2009

(51) Int. Cl. A61B 5/024; A61B 5/00; A61B 5/11; G10L 11/00

(52) U.S. Cl. 600/500; 600/300; 600/595; 704/270; 704/E11.001

(57) ABSTRACT

A medical device in accordance with the present invention comprises a connection device adapted to be wrapped around and securely attached to a patient, a data storage device attached to or integral with the connection device, wherein the data storage device comprises an input port adapted to receive medical data pertaining to the patient, a memory adapted to store the medical data, and an output port adapted to produce the medical data. A medical device in accordance with the present invention may also comprise a connection device adapted to be wrapped around and securely attached to a patient, a data storage device attached to or integral with the connection device, wherein the data storage device comprises a memory adapted to receive medial data pertinent to a patient, and a transceiver adapted to transmit and receive the medical data.

### TECHNICAL FIELD OF THE INVENTION

The present invention relates in general to a medical device, and more specifically, to a medical device utilized for the communication of medical data and the reduction of the likelihood of misidentification of medical patients.

### COPYRIGHT & TRADEMARK NOTICE

A portion of the disclosure of this patent application may contain material that is subject to copyright protection. The owner has no objection to the facsimile reproduction by any one of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and shall not be construed as descriptive or to limit the scope of this invention to material associated only with such marks.

### BACKGROUND OF THE INVENTION

It is common procedure at medical facilities for patients to wear identification bands. One of the main purposes of the identification band is to help reduce the chance of misidentification of a patient. Only if a patient is properly identified can medical personnel provide the necessary medical treatment. Should a patient be misidentified, the patient may be given the treatment intended for another and may not receive the care they need or require.

In order to properly utilize an identification band typically used in today's medical facilities, one must carefully read the information contained on the band. For medical personnel in a rush, or medical personnel with poor vision, it may be difficult to properly read the information contained on the identification band. Further, medical personnel must make sure the information contained on the identification band corresponds with the information contained in the patient's medical chart. Additionally, the patient may be incapacitated or unable to speak, and thus unable to notify the medical personnel should a misidentification arise.

The misidentification of patients in a hospital can often lead to improper and unnecessary medical procedures, in addition to expensive litigation and malpractice claims. Should a patient be misidentified, they may not receive urgent care that may be required. Further, should the patient have any allergies or other important medical concerns, conflicts or complications in treatment may arise, creating potential harm to the health and care of the patient.

Inventions, systems and methods have been developed for the proper identification of patients. These systems and methods, however, do not guarantee that mistakes will not be made. Current systems and methods for patient identification require the consultation of multiple sources of information, thereby increasing the workload required for medical personnel and increasing the likelihood of mistake.

Thus, there is a need in the art for a medical device that helps properly identify patients and communicate important medical communication through audible identification and confirmation methods. There is also a need in the art for a medical device that may measure, record and transfer medical data to an external electronic device. Such a device may help prevent the occurrence of errors or mistakes in a hospital or other medical location due to lack of attention, oversight, stress, or negligence. Specifically, there is a need for a medical device that will increase the efficiency and quality of medical care and reduce the likelihood of patient misidentification by medical personnel. It is to these ends that the present invention has been developed.

### SUMMARY OF THE INVENTION

To minimize the limitations in the prior art, and to minimize other limitations that will be apparent upon reading and understanding the present specification, the present invention describes a medical device utilized for the reduction of the likelihood of misidentification of medical patients.

A medical device in accordance with the present invention comprises a connection device adapted to be wrapped around and securely attached to a patient, a data storage device attached to or integral with the connection device, wherein the data storage device comprises an input port adapted to receive medical data pertaining to the patient, a memory adapted to store the medical data, and an output port adapted to produce the medical data.

A medical device in accordance with the present invention may also comprise a connection device adapted to be wrapped around and securely attached to a patient, a data storage device attached to or integral with the connection device, wherein the data storage device comprises a memory adapted to receive medial data pertinent to a patient, and a transceiver adapted to transmit and receive the medical data.

It is an objective of the present invention to reduce the likelihood of the misidentification of a patient.

It is another objective of the present invention to increase the efficiency of the patient check-in process in a hospital.

It is still another objective of the present invention to audibly output medical data, biometric data, or a special message from the patient to medical personnel in proximity to a patient.

It is yet another objective of the present invention to transfer medical data from a medical device to an external electronic device.

It is yet another objective of the present invention to display transferred medical data from a medical device onto an external display screen or monitor.

It is yet another objective of the present invention to improve the quality and efficiency of medical data transfer, communication and retrieval in a medical facility.

Finally, it is an objective of the present invention to increase the quality of medical care received by patients at a medical facility.

These and other advantages and features of the present invention are described herein with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1(a) illustrates a top plan view of an exemplary embodiment of a medical device.

FIG. 1(b) illustrates a front elevational view of an exemplary embodiment of a medical device.

FIG. 1(c) illustrates a front elevational view of an exemplary embodiment of a medical device with data storage device detached from wrist band.

FIG. 2(a) illustrates a flow chart utilized by medical device for the input and storage of medical data.

FIG. 2(b) illustrates a flow chart utilized by medical device for the output or transmission of medical data.

FIG. 3(a) illustrates a block diagram of an exemplary embodiment of a data storage device utilizing audio input and audio output ports.

FIG. 3(b) illustrates a block diagram of an exemplary embodiment of a data storage device utilizing an audio input and a data transmission device.

FIG. 4(a) illustrates a block diagram of an exemplary embodiment of a data storage device utilizing a data transmission device for both data reception and transmission.

FIG. 4(b) illustrates a block diagram of an exemplary embodiment of a data storage device utilizing a data transmission device for data reception and an audio output.

FIG. 5(a) illustrates a block diagram of an exemplary embodiment of a data storage device utilizing a biometric sensor input and an audio output.

FIG. 5(b) illustrates a block diagram of an exemplary embodiment of a data storage device utilizing a biometric sensor input and a data transmission device for data transmission.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

In the following discussion of the present invention, the term “medical data” may include, but is not limited to, a patient's name, consent and waiver of liability, list of known allergens, medical history, treatment or surgical instructions, personal messages from the patient, location of patient within medical facility, pulse rate, temperature, body movement, or other medical diagnostic data important to the patient treatment process.

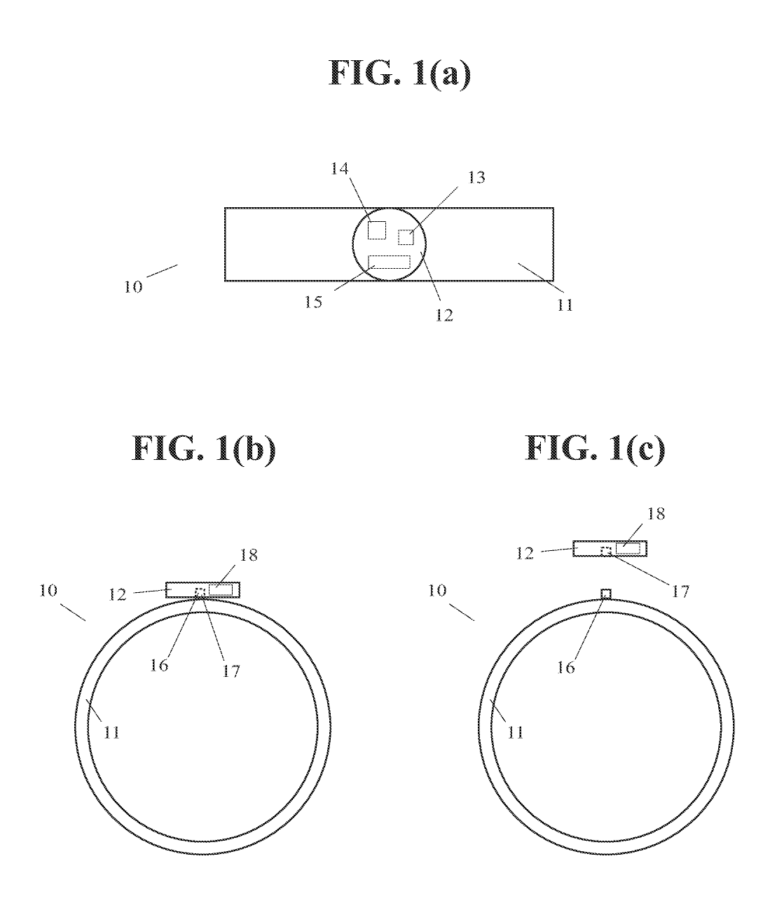

FIG. 1(a) illustrates a top plan view of an exemplary embodiment of a medical device. FIG. 1(b) illustrates a front elevational view of an exemplary embodiment of a medical device. FIG. 1(c) illustrates a front elevational view of an exemplary embodiment of a medical device with data storage device detached from wrist band.

FIGS. 1(a)-1(c) depict medical device 10, comprising connection device 11 and data storage device 12. Medical device 10 is designed to assist in the correct identification of a hospital patient and provide for an easy and efficient method of relaying accurate medical data and instructions to medical personnel throughout the medical treatment process, including but not limited to patient check-in, patient location, surgery preparation, medical examination, surgical operation, and post-operative care.

Connection device 11 is a component of medical device 10 that may be adapted to be wrapped around and securely attached to a patient. In an exemplary embodiment, connection device 11 may comprise a wristband and may be wrapped around and securely attached to a wrist of a patient. In other exemplary embodiments, however, connection device 11 may securely wrap around the wrist, leg, neck or other limb of a patient. Connection device 11 may securely wrap around or attach to patient by tie, mechanical snapping means, adhesive, elastic properties of the connection device material, or some other method of attachment. Further, in other embodiments of the present invention, connection device 11 may securely attach to a patient, or an article of clothing or other accessory on the patient, without wrapping around the patient's limb. Dimensions of connection device 11 may be tailored specifically to the designated limb of the patient, the size or diameter of the patient's limb, or connection device 11 may comprise a means for tightening or loosening to securely attach to a wrist of the patient.

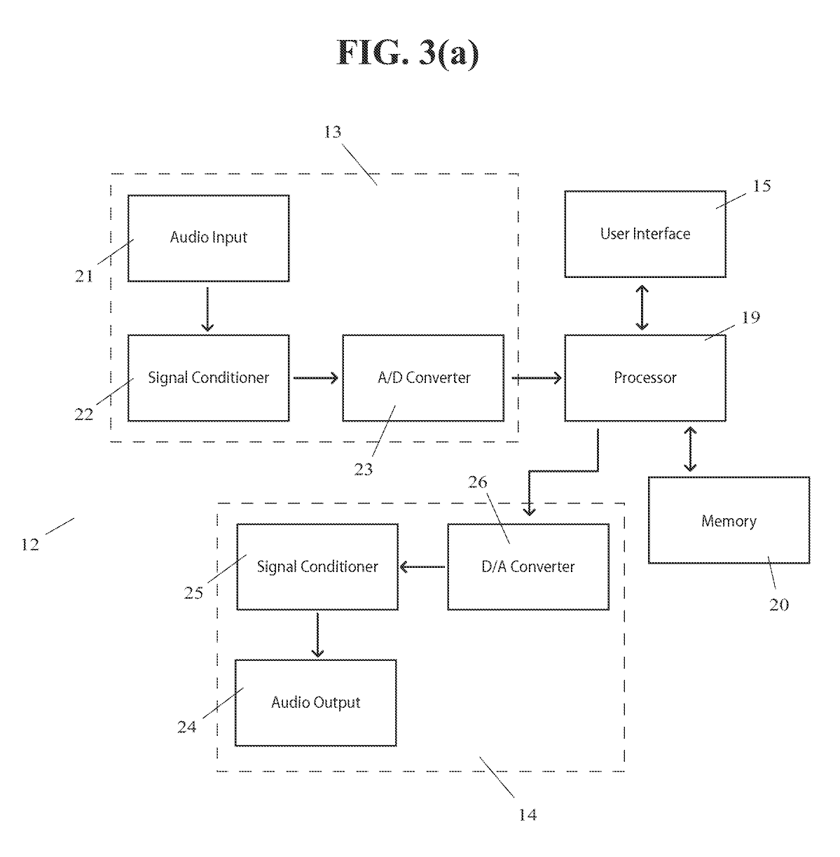

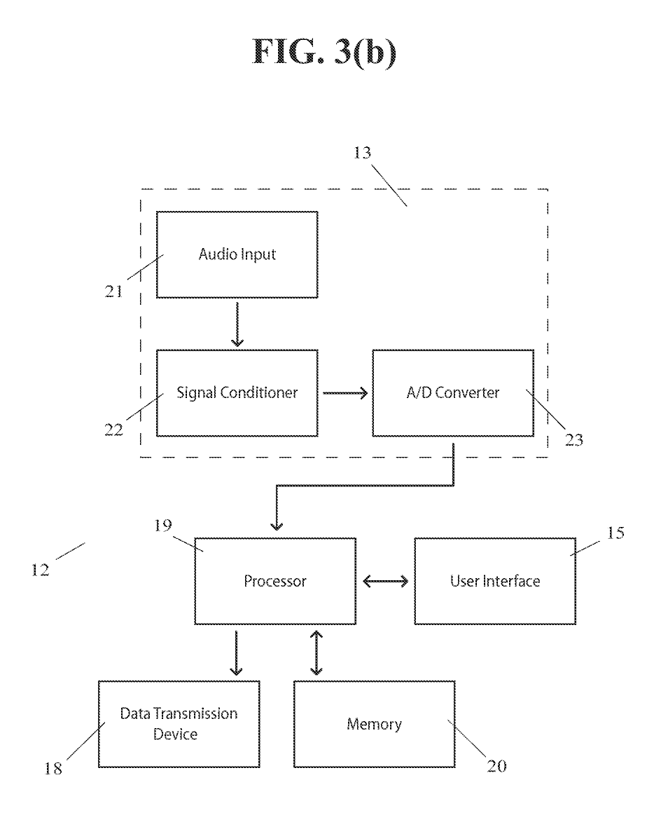

Data storage device 12 is designed to receive, store, and transmit or output patient medical data. Data storage device 12 may be permanently or removeably coupled to connection device 11, as illustrated in FIGS. 1(b) and 1(c). Data storage device 12 may comprise input port 13, output port 14 and memory 20 (see FIGS. 3-5, discussed below). Data storage device 12 may also comprise data transmission device 18 (illustrated in FIGS. 1(b) and 1(c)). In exemplary usage of data storage device 12, a user may input medical data to data storage device 12 via input port 13, wherein processor 19 (see FIGS. 3-5) may store medical data in memory 20, and said medical data may be audibly transmitted or spoken to the user through output port 14. Users of the present invention may include the patient, nurse, surgeon, medical receptionist, or any other hospital personnel authorized to control medical device 10 when in use by the patient.

Input port 13 is a component of data storage device 12 in which medical data is received. In various embodiments of the present invention, input port 13 may receive medical data in the form of audible speech or biometric data. In such embodiments, input port 13 may comprise of a microphone able to receive medical data in the form of audio speech or a biometric sensor capable of receiving biometric data. Additionally, data storage device 12 may comprise a plurality of input ports 13, located on the exterior housing of data storage device 12.

Output port 14 is a component of data storage device 12 through which medical data stored in data storage device 12 is outputted in the form of audible speech. Through user interface 15, a user may activate data storage device 12 to audibly output said medical data through output port 14. In an exemplary embodiment, user interface 15 may comprise a plurality of buttons on the exterior housing of data storage device 12 that, when activated by a user, medical data is “read” out to the user.

Data transmission device 18 is a component of data storage device 12 through which medical data may be transferred to or received from an external electronic device. In exemplary usage of the present invention, medical data may be transferred to medical device 10 via data transmission device 18 from an external electronic device. In further exemplary usage of the present invention, medical device 10 may receive medical data via data transmission device 18 from an external electronic device.

Illustrated in FIGS. 1(b) and 1(c), data storage device 12 may be removeably coupled to connection device 11 via connector 16 and receiver 17. In such embodiments, data storage device 12 may be reattachable to connection device 11. In an alternative embodiment of the present invention, however, data storage device 12 may be permanently attached to connection device 11. In all such embodiments, medical device 10 may be disposable or reusable. Connector 16 and receiver 17 may comprise components utilizing mechanical means, tie, screw rotation, or some other means of secure attachment to securely attach data storage device 12 to connection device 11.

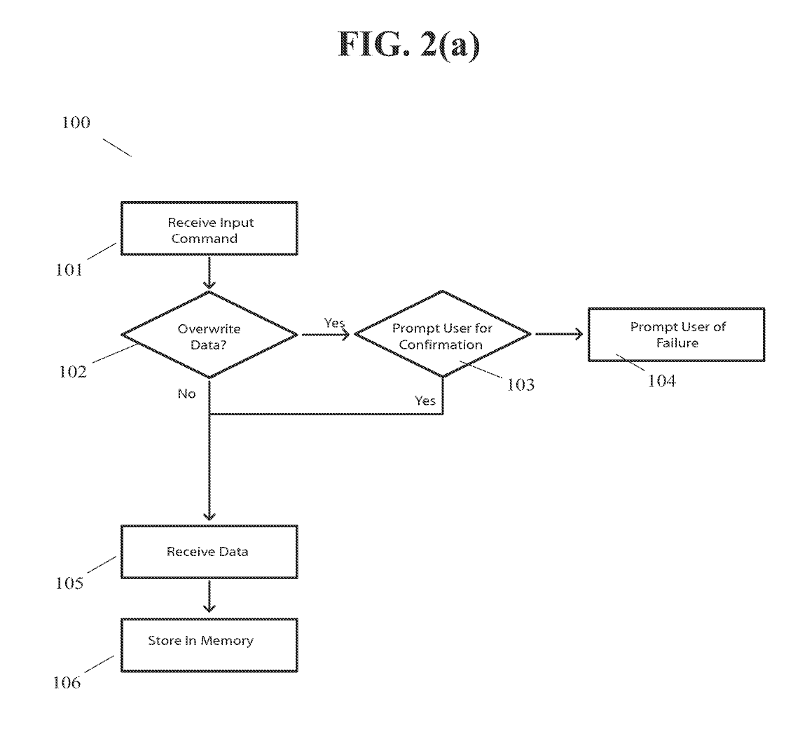

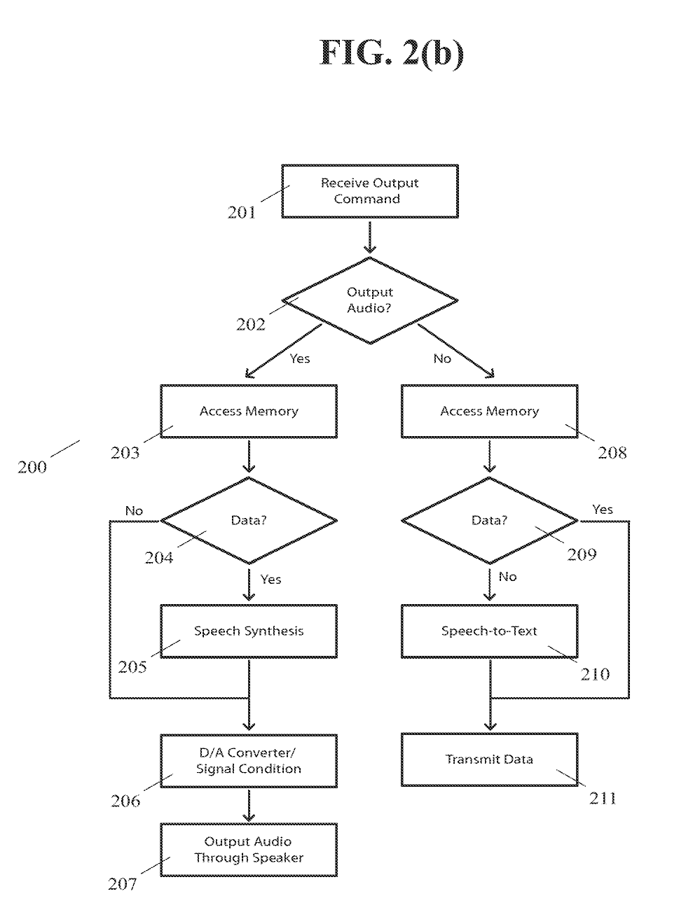

FIG. 2(a) illustrates a flow chart of sequence 100 utilized by medical device 10 for the input and storage of medical data. Sequence 100 is explained in the order described below; however, the following steps may be taken in any other conceivable sequence, or with additional steps not discussed, without deviating from the scope of the present invention.

In step 101, a user may activate medical device 10 through user interface 15 or via an external electronic device communication received through data transmission device 18. In an exemplary embodiment wherein user interface 15 may comprise a plurality of buttons or switches, or a touch screen interface, user interface 15 may be activated by pressing or activating said button, switch or touch screen. Alternatively, medical device 10 may be activated by a command signal sent from an external electronic device, received by data transmission device 18. Should medical device 10 be activated by a user, medical device 10 will then receive an input command and will proceed to step 102.

In step 102, processor 19 may determine if medical data need be overwritten in memory 20. If medical data has not already been stored in the designated storage slot in memory 20, then medical device 10 will proceed to step 105 because no medical data need be overwritten. If medical data has already been stored in the designated storage slot in memory 20, described below, then medical device 10 will proceed to step 103 because storage of new medical data would require old medical data to be overwritten. To determine if medical data need be overwritten, processor 19 checks the designated storage slot in memory 20 to see if medical data is already stored.

In step 103, the user is prompted for confirmation or administrative authentication. This prompt for confirmation may be done either through output port 14, user interface 15 or through an external electronic device via data transmission device 18. Should the user of medical device 10 provide confirmation through input port 13, user interface 15, or data transmission device 18, medical device 10 will proceed to step 105. If, however, confirmation is not provided by user, then medical device 10 will proceed to step 104.

In step 104, the user is prompted with a failure notice. Because confirmation for the deletion and overwriting of data was not provided, medical device 10 will not accept medical data transmission and store the medical data in memory 20. The user may be prompted with the failure notice either through output port 14, user interface 15, or via an external electronic device through data transmission device 18.

In step 105, medical device 10 will receive medical data, either through input port 13 or data transmission device 18. Once the medical data is received, medical device 10 will proceed to step 106. Finally in step 106, the medical data imported into medical device 10 is stored in memory 20.

FIG. 2(b) illustrates a flow chart of sequence 200 utilized by medical device 10 for the output or transmission of medical data. Sequence 200 is explained in the order described below; however, the following steps may be taken in any other conceivable sequence, or with additional steps not discussed, without deviating from the scope of the present invention.

In step 201, a user may activate medical device 10 through user interface 15 or via an external electronic device through data transmission device 18. In an exemplary embodiment of the present invention, user interface 15 may be activated by pressing or activating the button, switch or touch screen. Alternatively, medical device 10 may be activated by a command signal sent from an external electronic device, received by data transmission device 18. Should medical device 10 be activated by a user, medical device 10 will then receive an output command and will proceed to step 102.

In step 202, processor 19 may determine whether audio is to be outputted to the user of medical device 10, or whether medical data is to be transmitted to an external electronic device. Should speech be outputted to a user of medical device 10, medical device 10 will proceed to step 203. In the alternative, should processor 19 determine that medical data is to be transferred to an external electronic device, medical device 10 will proceed to step 208.

In step 203, processor 19 accesses the particular medical data stored in memory 20 that was requested in the output command. In step 204, processor 19 determines whether the medical data stored in memory 20 was stored as digital audio, or if the medical data is textual or otherwise non-audio in nature. If the medical data was stored as digital audio in memory 20, medical device 10 will proceed to step 206. If, however, the medical data was not stored as digital audio in memory 20, then medical device 10 will proceed to step 205 for speech synthesis.

In step 205, processor 19 performs a speech synthesis algorithm on the requested medical data to be audibly outputted to the user. In the speech synthesis algorithm, processor 19 converts the non-audio medical data into a format compatible for audio speech output. Once complete, medical device 10 will proceed to step 206.

In step 206, medical device 10 converts the digital medical data into an analog signal and conditions the analog signal. Finally, in step 207, medical device 10 outputs the medical data through the audio output of output port 14.

In step 208, processor 19 accesses the particular medical data stored in memory 20 that was requested in the output command. In step 209, processor 19 determines whether the medical data stored in memory 20 was stored as digital audio, or if the medical data is non-audio in nature. If the medical data was not stored as digital audio in memory 20, then medical device 10 will proceed to step 211. If, however, the medical data was stored as digital audio medical, then medical device 10 will need to perform a speech-to-text conversion algorithm in step 210.

In step 210, processor 19 performs a speech-to-text algorithm on the requested medical data to be digitally transmitted to an external electronic device. In the speech-to-text algorithm, processor 19 converts the audio medical data into a non-audio based format compatible for data transmission to an external electronic device. Once complete, medical device 10 will proceed to step 211. Finally, in step 211, medical device 10 transmits the converted medical data to an external electronic device via data transmission device 18.

A medical device in accordance with the present invention may further comprise a connection device and a data storage device, which may comprise audio input and output ports. FIG. 3(a) illustrates a block diagram of an exemplary embodiment of a data storage device utilizing audio input and audio output ports. FIG. 3(a) shows data storage device 12, comprising input port 13, output port 14, processor 19, and memory 20. Input port 13 may comprise audio input 21, signal conditioner 22, and analog to digital converter 23 (“A/D converter”), and output port 14 may comprise audio output 24, signal conditioner 25, and digital to analog converter 26 (“D/A converter”). In the embodiment of the present invention depicted in FIG. 3(a), data storage device 12 is designed to receive medical data from an audible source and to audibly output medical data.

Input port 13, which may comprise audio input 21, signal conditioner 22, and A/D converter 23, may receive medical data from an audible source and transform it into a digital form that may be stored in memory 20. In an exemplary process performed by data storage device 12 of receiving and storing medical data, audible medical data may be inputted by a user through audio input 21, which in an exemplary embodiment may comprise a microphone. An analog signal of the medical data may be amplified and cleaned by signal conditioner 22. Once conditioned, the medical data may be transformed into digital data by A/D converter 23. Processor 19 may then store the medical data in memory 20.

Output port 14, which comprises audio output 24, signal conditioner 25, and D/A converter 26, may transform the medical data stored in memory 20 into an analog signal then audibly output the medical data to a user. In an exemplary process performed by data storage device 12, medical data stored in memory 20 may be converted into an analog signal by D/A converter 26. The analog signal of the medical data may then be amplified and cleaned by signal conditioner 25. Once conditioned, the analog signal of the medical data may be audibly dispatched to the user through audio output 24, which in an exemplary embodiment may comprise a speaker.

Processor 19 is a controlling component of data storage device 12. In an exemplary embodiment of the present invention, when a user activates user interface 15 to input medical data, processor 19 activates input port 13 in order to turn on audio input 21. Processor 19 may also store digital medical data within memory 20. Further, when user interface 15 is activated to output medical data, processor 19 may access memory 20 and transfer it to output port 14 in order to dispatch audible medical data.

In an exemplary embodiment of the present invention, memory 20 is non-volatile and electrically erasable programmable read-only memory (EEPROM), allowing data to be stored when power is removed from medical device 10. In alternative embodiments of the present invention, however, memory 20 may comprise another memory type or format.

In one embodiment of the present invention, medical data may be stored in memory 20, but may not be overwritten by any subsequent input. In alternative embodiments of the present invention, however, medical data may be stored in memory 20 and later overwritten with new medical data. In such embodiments, a user may input and overwrite medical data in memory 20, or the user may be required to input an administrative authorization or be prompted for confirmation in order to overwrite the stored medical data as described in sequence 100.

In exemplary embodiments of the present invention, memory 20 may contain a specific number of storage slots for medical data. In such embodiments, each specific storage slot in memory 20 may be designated for a specific piece or type of medical data. For example, in one such embodiment, slot one of memory 20 may be designated for an audio recording of the patients name, while slot two may be designated for an audio recording of the patients consent to surgery. In such an embodiment, the amount of medical data that may be stored in data storage device 12 is limited by both the number of slots available and the total storage capacity of memory 20. In alternative embodiments of the present invention, however, memory 20 may be dynamic in nature, meaning that slots are not defined in size or quantity, and the amount of medical data stored is only limited by the total storage capacity of memory 20.

A medical device in accordance with the present invention may alternatively comprise a connection device and a data storage device, which may comprise audio input port and a data transmission device. FIG. 3(b) illustrates a block diagram of an exemplary embodiment of a data storage device utilizing an audio input and a data transmission device. FIG. 3(b) shows data storage device 12, comprising input port 13, data transmission device 18, processor 19, and memory 20. In the embodiment of the present invention depicted in FIG. 3(b), data storage device 12 is designed to receive medical data from an audio input, and transmit stored medical data to an external electronic device via data transmission device 18.

In the embodiment of medical device 10 depicted in FIG. 3(b) a user may activate audio input and medical data storage via user interface 15. Alternatively, a user may activate audio input and medical data storage via an external electronic device through data transmission device 18.

Data transmission device 18 is a component of medical device 10. In the embodiment of medical device 10 depicted in FIG. 3(b), data transmission device 18 may transmit medical data stored in memory 20 to an external electronic device. In the present embodiment, data transmission device 18 may transmit medical data to an external electronic device. In alternative embodiments of the present invention, data transmission device 18 may accept command instructions from an external electronic device.

In the present embodiment, medical data may be audibly inputted to medical device 10 via input port 13 and stored in memory 20 as a digital audio recording. Depending upon the command instruction inputted into medical device 10, the audio medical data may be transmitted via data transmission device 18, or alternatively, processor 19 may perform a speech-to-text algorithm, creating text data from the digital audio recording, thereby allowing textual medical data to be transferred via data transmission device 18 to the external electronic device. As such, data transmission device 18 may transmit either digital audio medical data or textual medical data to an external electronic device.

Data transmission device 18 may comprise a wired connection interface. In an embodiment comprising a wired connection interface, data transmission device 18 may comprise a universal serial bus (“USB”) connection interface such that medical device 10 may couple with an external electronic device via a USB cable and transmit medical data to, and receive medical data from said external electronic device. Alternatively, in another exemplary embodiment, data transmission device 18 may comprise a fire wire connection interface.

In other embodiments of the present invention, data transmission device 18 may comprise a wireless connection interface. In such embodiments, data transmission device 18 may utilize peer-to-peer or network based wireless protocols such as Bluetooth, wifi, cellular, analog, or some other type of wireless data transmission technology protocol. In such embodiments, medical device 10 may transmit medical data to, or receive medical data from, an external electronic device via wireless transmission to or from data transmission device 18.

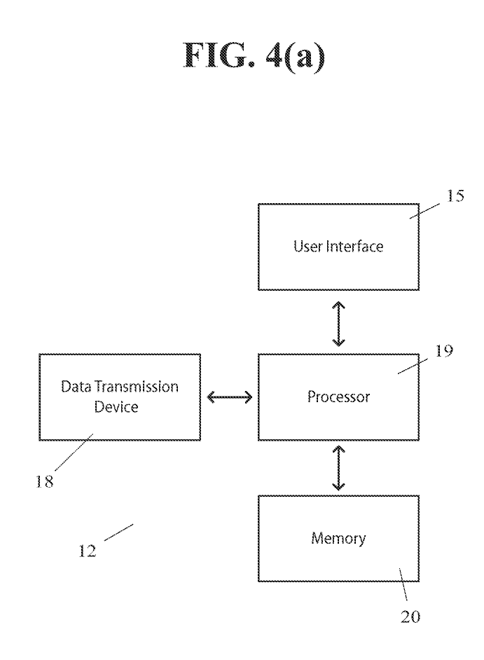

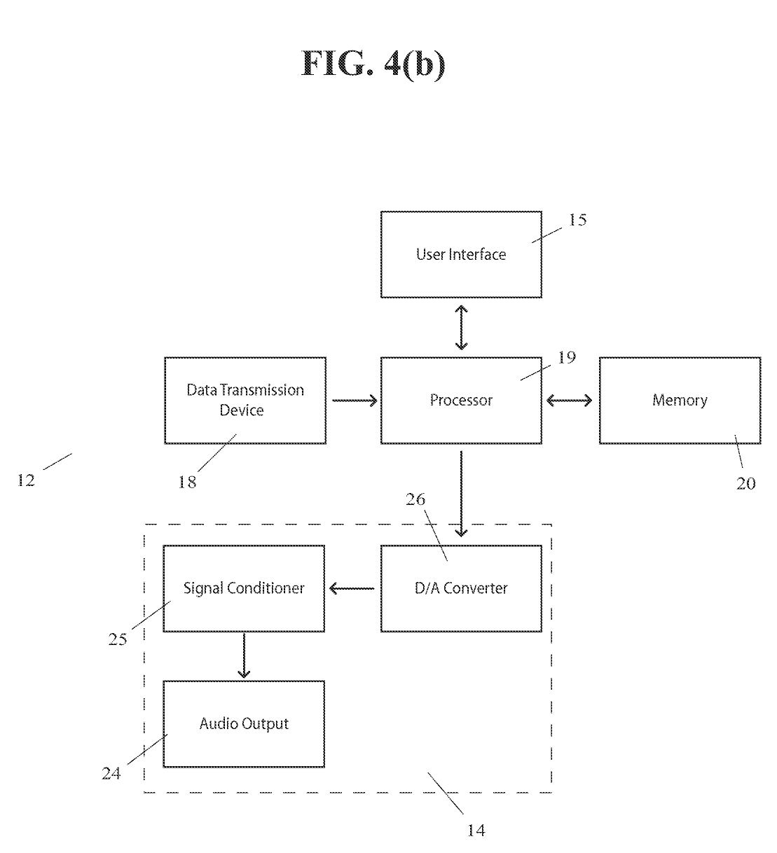

Other embodiments of medical device 10 may comprise a data transmission device for the transmission and reception of medical data. FIG. 4(a) illustrates a block diagram of an exemplary embodiment of a data storage device utilizing a data transmission device for both data reception and transmission. FIG. 4(a) shows data storage device 12, which comprises data transmission device 18, processor 19, and memory 20. In the embodiment of medical device 10 depicted in FIG. 4(a), medical data may be transmitted from or received by data transmission device 18.

In the embodiment of the present invention depicted in FIG. 4(a), medical data may be transmitted from an external electronic device to medical device 10 via data transmission device 18 and stored in memory 20. Medical data may also be transmitted to an external electronic device from medical device 10 via data transmission device 18. Medical data transmission or reception may be activated by a user command input into user interface 15 or from an external electronic device received via data transmission device 18.

Alternatively, medical data may be output by medical device 10 through user interface 15. In an exemplary embodiment of the present invention, user interface 15 may comprise a visual display such that medical data may be visually outputted to a user. In another embodiment, user interface 15 may comprise a touch screen visual display such that the user may input commands to medical device 10 through the touch screen, and medical data may be displayed.

Medical device 10 may also audibly output medical data that was received through electronic data transmission. FIG. 4(b) illustrates a block diagram of an exemplary embodiment of a data storage device utilizing a data transmission device for data reception and an audio output. FIG. 4(b) shows data storage device 12, comprising data output 14, data transmission device 18, processor 19, and memory 20. Data output 14 may further comprise audio output 24, signal conditioner 25, and D/A converter 26. In the present embodiment, medical data may be received through data transmission and audibly outputted to a user.

In the embodiment of the present invention depicted in FIG. 4(b), medical data may be audibly outputted to a user whether it is stored in speech or textual form. Should medical data be imported through data transmission device 18 in textual or otherwise non-audible form, processor 19 must perform a text-to-speech algorithm, thereby converting the medical data into a format compatible with audible output. If the medical data imported is already in an audible output format, text-to-speech need not be performed. Once the medical data is in an audio output-compatible format, processor 19 may instruct output port 14 to output the medical data through audio output 24, as previously described (see discussion above, FIG. 3(a)).

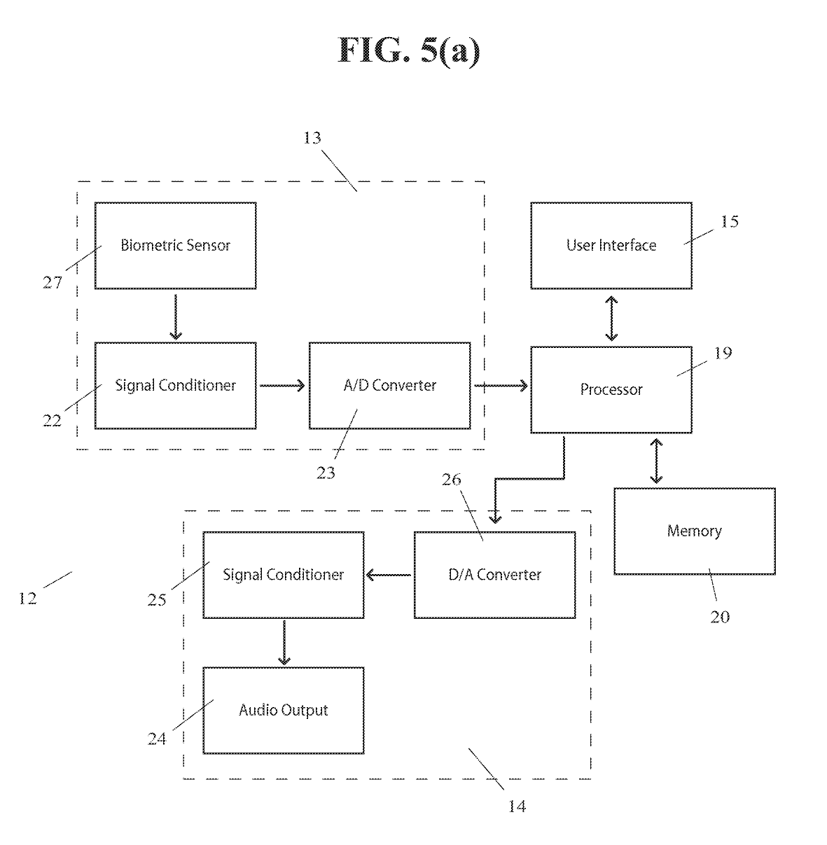

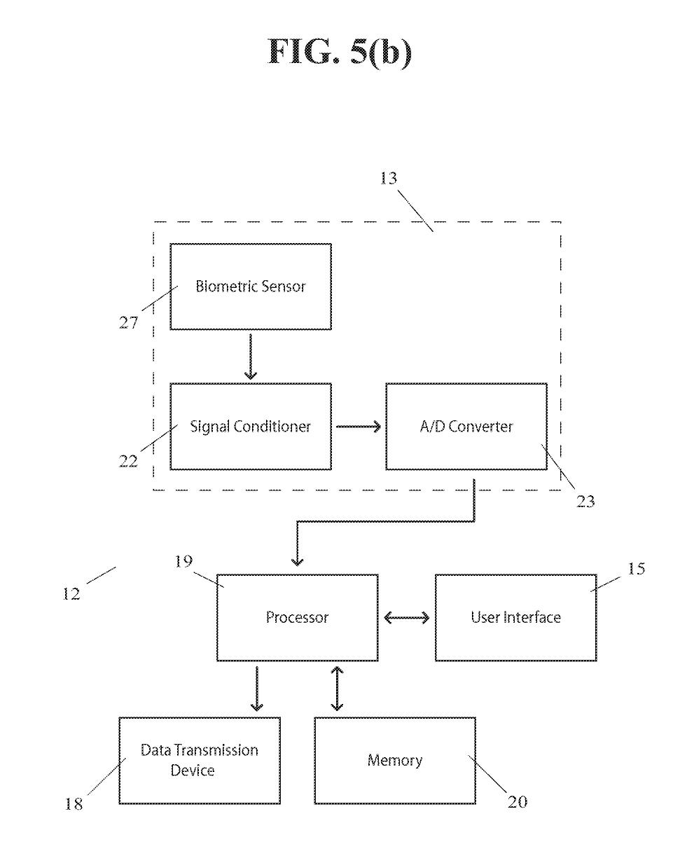

A medical device in accordance with the present invention may comprise a biometric sensor for medical data recording and input. FIG. 5(a) illustrates a block diagram of an exemplary embodiment of a data storage device utilizing a biometric sensor input and an audio output. FIG. 5(b) illustrates a block diagram of an exemplary embodiment of a data storage device utilizing a biometric sensor input and a data transmission device for data transmission. FIGS. 5(a) and 5(b) show data storage device 12, comprising input port 13, processor 19, memory 20, and output port 14 or data transmission device 18. Input port 13 may further comprise biometric sensor 27, signal conditioner 22, and A/D converter 23. In the embodiment of the present invention depicted in FIGS. 5(a) and 5(b), medical device 10 may receive biometric medical data and output the medical data audibly or through data transmission.

Biometric sensor 27 is a component of input port 13 in an exemplary embodiment of medical device 10. Biometric sensor 27 may sense and record a variety biometric medical data types. For example, in one embodiment of the present invention, if medical device 10 is worn on the surface of the patient's body, biometric sensor 27 may record the pulse rate of a patient. Alternatively, biometric sensor 27 may measure and record the temperature of a patient, the breathe rate of a patient, movement of a patient, or some other biometric medical data type.

Once biometric medical data is recorded by biometric sensor 27, the medical data is conditioned and digitally converted by signal conditioner 22 and A/D converter 23, respectively. Processor 19 may then store the biometric medical data in memory 20, where it later may be accessed and outputted audibly by output port 14 or transferred digitally by data transmission device 18.

The utilization of biometric sensor 27 may also allow medical device 10 to provide audio or visual alerts to medical personnel should the biometric medical data trigger some predetermined activation mechanism. For example, where medical device 10 is utilized to monitor a patient, and biometric sensor 27 is actively recording the patient's pulse rate, should the patient's pulse reach a certain predetermined level, an audio alert may be triggered by processor 19 and outputted through output port 14. Such an audible warning may be outputted by audio output 24, including a speech output stating “warning,” “high pulse rate,” or “attention,” or a reading of the pulse rate.

Alternatively, medical device 10 may be utilized to provide for alerts to be transmitted to an external electronic device should a predetermined triggering mechanism be activated. For example, where medical device 10 is utilized to monitor a patient, and biometric sensor 27 is actively recording the patient's body temperature, an alert may be transmitted to an external electronic device to alert medical personnel when the patient's body temperature reaches a specific level. Such a data transmission may be transmitted by data transmission device 18 and may include an audio or visual alert on an external electronic device, including screen flashing, beeper alarms, text messages, hospital P.A. announcement, or some other external electronic device activation.

What is claimed is:

1. A medical device, comprising:
a connection device adapted to be wrapped around and securely attached to a patient;
a data storage device attached to or integral with the connection device, wherein the data storage device comprises:
an input port adapted to receive medical data pertaining to the patient;
a memory adapted to store the medical data; and
an output port adapted to produce the medical data.

2. The medical device of claim 1, wherein the input port comprises:
a microphone adapted to convert speech into an audio signal for audio input;
a signal conditioner adapted to process the audio signal; and
an analog to digital converter adapted to digitize the processed audio signal to generate the medical data.

3. The medical device of claim 1, wherein the input port comprises:
a biometric sensor adapted to generate a biometric signal;
a signal conditioner adapted to process the biometric signal; and
an analog to digital converter adapted to digitize the processed biometric signal to generate the medical data.

4. The medical device of claim 3, wherein the biometric sensor is adapted to measure the temperature of the patient.

5. The medical device of claim 3, wherein the biometric sensor is adapted to measure the pulse rate of the patient.

6. The medical device of claim 3, wherein the biometric sensor is adapted to measure movement of the patient's limb.

7. The medical device of claim 1, wherein the output port comprises:
a digital to analog converter adapted to convert digitized audio data based on the medical data into an analog audio signal;
a signal conditioner adapted to process the analog audio signal; and
a speaker for audio output adapted to produce sound based on the processed analog audio signal.

8. The medical device of claim 1, wherein the data storage device is comprises a receiver adapted to connect to a corresponding connector on the connection device.

9. The medical device of claim 1, wherein medical data comprises one or more of the following:
information related to a patient;
information related to the patient's waiver;
information related to the patient's consent;
information related to patient allergies;
information related to special surgical instructions; or
information related to message to medical personnel;

10. The medical device of claim 1, further comprising a processor for receiving medical data and storing in memory.

11. The medical device of claim 1, wherein the input port comprises a wireless peer-to-peer connection interface.

12. A medical device, comprising:
a connection device adapted to be wrapped around and securely attached to a patient;
a data storage device attached to or integral with the connection device, wherein the data storage device comprises:
a memory adapted to receive medial data pertinent to a patient; and
a transceiver adapted to transmit and receive the medical data.

13. The medical device of claim 12, wherein the transceiver comprises a Bluetooth device adapted to transmit and receive medical data.

14. The medical device of claim 12, wherein the transceiver comprises a USB connection interface adapted to transmit and receive medical data.

15. The medical device of claim 12, wherein the transceiver comprises a wireless local area network connection interface adapted to transmit and receive medical data.

16. The medical device of claim 12, wherein the transceiver comprises a cellular connection interface adapted to transmit and receive medical data.

17. The medical device of claim 12, wherein the data storage device further comprises an input port adapted to receive medical data pertaining to the patient, comprising:
a microphone adapted to convert speech into an audio signal for audio input;
a signal conditioner adapted to process the audio signal; and
an analog to digital converter adapted to digitize the processed audio signal to generate the medical data.

18. The medical device of claim 12, wherein the data storage device further comprises an input port adapted to receive medical data pertaining to the patient, comprising:
a biometric sensor adapted to generate a biometric signal;
a signal conditioner adapted to process the biometric signal; and
an analog to digital converter adapted to digitize the processed biometric signal to generate the medical data.

19. The medical device of claim 12, wherein the data storage device further comprises a digital to analog converter adapted to convert digitized audio data based on the medical data into an analog audio signal;
a signal conditioner adapted to process the analog audio signal; and
a speaker for audio output adapted to produce sound based on the processed analog audio signal.

20. The medical device of claim 12, further comprising a processor for receiving medical data and storing in memory.
